(19) United States
(12) Patent Application Publication
(10) Pub. No.: US 2011/0293748 A1
(43) Pub. Date: Dec. 1, 2011

(54) COMBINED PREPARATION FOR TREATING SEPSIS

(75) Inventor: Thomas STIEFEL, Stuttgart (DE)

(73) Assignee: Biosyn Arzneimittel GMBH, Fellbach (DE)

(21) Appl. No.: 13/207,864

(22) Filed: Aug. 11, 2011

Related U.S. Application Data
(62) Division of application No. 10/575,419, filed on Jan. 10, 2007.

(30) Foreign Application Priority Data
Oct. 22, 2003 (DE) 10349115.5

(51) Int. Cl. A61K 33/04; A61P 29/00; A61P 7/00

(52) U.S. Cl. 424/702

(57) ABSTRACT

The present invention relates to a method of treating sepsis, systemic inflammatory response syndrome (SIRS), and/or septic shock in a patient. The method comprises administering (a) a pharmaceutical composition comprising a selenium-containing active substance, (b) a pharmaceutical composition comprising a corticoid-containing active substance, and (c) a pharmaceutical composition comprising insulin.

### CROSS REFERENCE TO RELATED PATENT APPLICATIONS

This application is a divisional of copending U.S. patent application Ser. No. 10/575,419, filed Jan. 10, 2007, which is the U.S. national phase of International Patent Application PCT/EP2004/011978, filed on Oct. 22, 2004, which claims priority to German Patent Application No. 103 49 115.5-41, filed Oct. 22, 2003, all of which are hereby incorporated by reference.

The present invention relates to a combined preparation for treating sepsis.

Sepsis, SIRS (systemic inflammatory response syndrome) and septic shock are the main causes of death in non-cardiological intensive care units. According to the Center of Disease Control, in the USA, approx. 200,000 persons annually die as a consequence of sepsis, comparable to the mortality rate due to myocardial infarction. According to up-to-date investigations in the USA, the number of sepsis cases increased from 1979 with 82.7/100,000 to 240/100,000 in the year 2000. The prevalence of sepsis in the USA is estimated to be approx. 600,000 per year. With an incidence of approx. 300 per 100,000 inhabitants, sepsis is a more frequent disease than intestine cancer (50/100,000), breast cancer (110/100,000) or Aids (17/100,000). In the period from 1979 to 2000, the mortality rate decreased from 27.8% to only 17.9%, and thus the number of patients dying from sepsis has significantly increased within the last 20 years. The estimated hospital costs amount to approx. 17 thousand billions US$. Despite the development of very effective antibiotics, the mortality rate of sepsis patients could not be essentially influenced by their broad application. Obviously, it is not the micro-organisms alone which are responsible for the deadly course, but also the reaction of the organism to the infection. There is a large agreement that the overstimulation of the immune system by the cytokines activated in the sepsis results in multiorgan failure and death. Intervention studies blocking the reaction resulting from cytokines, such as TNFα, did in any way not lead to an improved survival.

Sepsis is the body's answer to an infection. Normally, the body's own immune system combats an infection, but in case of a severe sepsis, the reaction of the body leads to an overshooting and starts a cascade of processes leading to an expanded inflammation and blood clotting in tiny vessels in the whole body. The forms of sepsis also include severe sepsis which occurs if it comes to an acute organ dysfunction or a complete organ failure; septic shock arising in case of a severe sepsis if the cardiovascular system starts failing such that the blood pressure drops and vital organs are no longer provided with an adequate amount of oxygen.

The cause of a sepsis can be any infection—of bacterial, viral, parasitic origin or caused by fungi—and this infection can occur anywhere in the body. Sepsis can affect anybody at any age, although young or very old hospital patients and persons with existing disease conditions have a higher risk. Risk factors include a too little active immune system (as can arise, for example, during a chemotherapy or is caused by medicine intended to permit an organ transplantation; by surgical procedures; artificial respiration; genetic predisposition or in invasive procedures, such as the supply of liquids.)

Sepsis is the body's answer to an infection. The symptoms can include the following: fever and ague; decreased mental attention, sometimes in combination with confusion; diarrhoea; increased pulse frequency (more than 90 pulses per minute); increased respiration frequency (more than 20 breathes per minute); high or low degree of white platelets; low blood pressure; altered kidney or liver function. A sepsis can develop quickly. The earlier it is diagnosed and treated, the better. The most frequent infection sites leading to sepsis are the lung, the intestinal tract, the abdomen, and the pelvis. With up to 30% of the patients, however, the precise cause of infection is not identified. The course of the disease can often be unpredictable.

The course of the sepsis can be described as a sequence of various processes. When the sepsis begins, the body reacts with expanded inflammations, blood clotting and impaired decomposition of blood clots.

Under normal circumstances, substances, also referred to as immunomodulators, are released in order to support the body in combatting the infection during the healing process itself. With a person suffering from sepsis, this mechanism breaks down and the immunoregulators lead to an overshooting reaction. The infection enhances the release of too many of these regulators which inflame the lining of the blood vessels and the processes for blood coagulation are activated, this process triggering another wave releasing regulators. The inflammation leads to the release of a substance stimulating the formation of blood clots. In the sepsis cascade, the body's ability to decompose the blood clots is suppressed. A substance involved in the formation of the blood clot, the control of the inflammation and the decomposition of clots, referred to as activated protein C, is reduced in a sepsis. As a result of the formation of blood clots and the inability to decompose these clots, microscopic blood clots start to deposit in vital organs, arms and legs and restrict the blood flow leading to tissue damages which can lead to organ failure.

The diagnosis of sepsis can sometimes be difficult. Some symptoms, such as fever, high pulse and respiratory difficulties, often occur and can sometimes be attributed to another cause. The first measure in the treatment is the identification and removal of the underlying infection with infection-inhibiting means or surgical procedures in order to remove the focus of infection. Depending on the condition of the patient, other treatments can be performed, such as the administration of liquids, active substances for increasing blood pressure, mechanical respiration in order to support respiration or dialysis in case of kidney failure.

Until recently, no means and no treatment strategy at all showed sufficient effect for the routine treatment of patients suffering from sepsis.

For example, the use of gluconates, such as the Mg gluconate, has the principal disadvantage of intervening in the blood sugar regulating mechanism of the body. Therefore, principally, but in particular in case of an additional use of insulin, the use of such substances should be avoided in sepsis/SIRS treatment.

Some physicians suppose that an active substance which could increase activated protein C, could be the key to a successful treatment of severe sepsis if the risk of dying is very high.

For example, the use of gluconates, such as the Mg gluconate, has the principal disadvantage of intervening in the blood sugar regulating mechanism of the body. Therefore, principally, but in particular in case of an additional use of insulin, the use of such substances should be avoided in sepsis/SIRS treatment.

Free radicals are also a possible target in the treatment of sepsis.

Free Radicals and Sepsis

It is well-known that during a systemic inflammation or sepsis hydrogen peroxide and superoxides are released. Simultaneously, however, the antioxidative mechanisms, such as the activity of superoxide dismutase, glutathione peroxidase (GPx) and catalase as well as the concentration of α-tocopherol and ascorbic acid are reduced. The increased expression of the iNOS (inducible nitric oxide synthase) causes a vasodilatation and translocation of NF-κB and thus the transcription and translatation of a number of inflammatory cytokines. NO reacts with the superoxide radicals to tissue-toxic peroxynitrite. The cell damage caused by free radicals can be detected by means of the increased levels of conjugated diens, thiobarbituric acid reaction products and malonic dialdehydes with SIRS and sepsis.

An adjuvant therapy with antioxidants, such as ascorbic acid, glutathione, N-acetyl-L-cysteine or vitamins A, E and C alone or in combination can reduce the morbidity of patients having severe burns. Moreover, the micro-circulation can be improved, the lipid peroxidation can be reduced, the heart minute volume can be increased and thus the whole volume substitution can be reduced. The translocation of NF-κB is lower, thereby less inflammatory cytokines, such as TNFa, IL-1β and IL-6 are released. The free radicals increasingly formed with SIRS/sepsis are also very important in organ damaging, and a therapy with antioxidants has a favourable influence on the natural course of a sepsis by modulation of the immunoreaction.

Selenoenzymes, the glutathione reductases (GPx) and thioreductases are the central enzyme systems maintaining the redox equilibrium plasmatically, cytosolically as well as in the cell core. They require selenium for forming the 21st amino acid, selenocysteine, which is present in the active centre of the selenium enzymes.

The plasma selenium levels are significantly reduced with patients having SIRS/sepsis. It is true that they do not reflect the selenium content of the organism, however, the simultaneously reduced plasmatic GPx-activity shows that obviously the demand of selenium is increased in the sepsis. Although one could not demonstrate by now that all tissue-resident selenoenzymes involved in the redox system are reduced, it could be nevertheless be proved that the NF-κB translocation is reduced under a selenium substitution which indicates that obviously less free radicals are formed.

The active principle of antioxidants is the inhibition of the formation (deferoxamon, alopurimol) or binding (radical scavenger, N-acetyl cysteine, dimethyl sulfoxide, dimethyl sulphur urea) and the decomposition (superoxide dismutase, catalase) of oxygen radicals.

It showed that in patients with a developing respiratory insufficiency in the course of a sepsis, N-acetyl cysteine lead to an improvement of the pulmonary function as well as an improvement of the radiological changes.

If vitamin E is employed, the lipid peroxidation in the sepsis is reduced. Reduced vitamin E serum levels have been detected more frequently in sepsis-induced ARDS cases.

The administration of vitamin C can also dramatically reduce serum lipid peroxides. Recently, in particular tocopherol and ascorbic acid have been examined as possible active substances in sepsis.

The present invention was based on the technical problem of providing another pharmaceutical preparation permitting an improvement of existing therapies in the treatment of sepsis or SIRS or septic shock, respectively. In particular, the problem was to provide a means which can reduce the considerably high morbidity in sepsis cases.

This technical problem is solved according to the invention by a pharmaceutical composition, containing a combination of active substances comprising a selenium containing active substance and a corticoid.

The mentioned active substances do not necessarily have to be present together in one form of administration, but they can be employed as single formulations. The administration of the two active substances can be effected either simultaneously or at intervals. In a preferred embodiment, the combination of active substances of selenium and corticoid is additionally supplemented by insulin, insulin providing a supporting function such that a strict blood sugar adjustment with patients suffering from a severe sepsis causes an altogether positive course of the disease. This employment of insulin can also be considered as a therapy supporting the actual therapy with the active substances selenium and corticoid.

The formulation of the active substances to an appropriate preparation is known to the person skilled in the art and can, for example, also be taken from European Pharmacopoeia, 4th edition, supplement 4.6, published by the EDQM, 2003. The active substances, as they are already commercially known as individual preparations, can be easily combined to provide the combination of active substances according to the invention.

In another preferred embodiment, the active substances are each present in an aqueous solution, and this solution should be preferably appropriate for the i.v. application of the active substances.

Appropriate concentrations of the selenium-containing active substance range from 5-500 μg/ml selenium. However, it should be noted that these data refer to the actual part by weight of the selenium, which is, of course, lower in case of sodium selenite than it would be in case of pure selenium. Accordingly, 50 μg selenium correspond to 0.167 mg sodium selenite×5H₂O. The selenium-containing active substance is preferably selected from pharmaceutically acceptable selenium salts, sodium selenite being employed as particularly preferred selenium-containing active substance.

In another preferred embodiment, the corticoid is selected from glucocorticoids.

As a particularly preferred embodiment, the pharmaceutical composition according to the invention contains hydrocortisone (cortisol) as corticoid ingredient.

The concentration of the corticoid ingredient preferably ranges from 0.5-50 mg/ml. Particularly preferred is 5 mg hydrocortisone/ml in a 50% ethanol-water mixture (v/v).

The person skilled in the art will select or tune corresponding additives, carriers, diluents, etc. of the pharmaceutical composition depending on the form of administration. Such pharmaceutical compositions differ from hydrocortisone compositions which are, for example, employed in the form of topically administrable preparations in ophthalmology.

The combination of active substances according to the invention is preferably employed for treating sepsis, SIRS or septic shock. In particular, the systemic reaction to an infection is considered to be a “sepsis”, which reaction is characterised by two or more of the following symptoms:

a. body core temperature>38° C. or <36° C.,
b. heart frequency>90/min
c. respiration frequency>20/min or PaCO₂<32 mm Hg (spontaneous respiration),
d. leukocytes>12,000/mm³ or <4,000/mm³, or >10 immature (rod-shaped) forms,

wherein in case of a “severe sepsis”, additionally an organ dysfunction, insufficient perfusion or hypotonia occurs. The insufficient circulation or circulatory disturbances can include a lactate acidosis, oliguria or an acute change of the state of awareness. These symptoms also involve the “sepsis syndrome”, which is also characterised by a systemically inflammatory reaction to an infection and comprises two or more of the following symptoms:

a. body core temperature>38° C. or <36° C.,
b. heart frequency>90/min,
c. respiration frequency>20/min or PaCO₂<32 mm Hg (spontaneous respiration)
d. at least one of the following indications of an insufficient organ function/organ perfusion:
altered cerebral function (disturbed state of awareness)
PaO₂<75 mm Hg (in room air, no COPD existing)
increased serum lactate concentration
reduced HTV: <30 ml/h or <0.5 ml/kg* for more than 1 h

“SIRS” means a systemic-inflammatory reaction to various severe clinical insults, which are also characterised by two or more of the following clinical symptoms:

a. body core temperature>38° C. or <36° C.,
b. heart frequency>90/min
c. respiration frequency>20/min or PaCO₂<32 mm Hg (spontaneous respiration),
d. leukocytes>12,000/mm³ or >4,000/mm³, or 10% immature (rod-shaped) forms.

Finally, a “septic shock” is a sepsis-induced shock with hypotonia despite adequate volume substitution in combination with circulatory disturbances. A special form is the “refractory septic shock” which is a septic shock without quick response to intravenous volume administration (for example, to 500 ml plasma expander in 30 minutes) and vasopressor (for example dopamine, more than 10 μg/kg per minute).

In a preferred embodiment, at least 100 μg, preferably at least 500 μg selenium (corresponding to, for example, 1.67 mg sodium selenite×5 H₂O) are administered per day. In a particularly preferred embodiment, at least 3.34 mg sodium selenite are administered per day (corresponding to 1000 μg selenium).

A preferred form of administration of the selenium is the administration by means of a single bolus injection per day.

Further preferred forms of administration are parenteral (i.v.) and oral administrations. Where circumstances require, the person skilled in the art will furthermore employ an enteral administration (e. g. via stomach or intestinal tube).

In another preferred embodiment, the administration of the selenium-containing active substance is performed for a duration of at least 7 days, preferably for a duration of at least 14 days.

In another preferred embodiment, an additional basis application of selenium is performed, for example at least 20 μg, preferably at least 35 μg sodium selenite per day. This additional basis application serves for balancing the usual loss in case of a total parenteral nutrition.

In another preferred embodiment, at least 50 mg hydrocortisone are administered per day, an amount of 200 mg hydrocortisone per day being particularly preferred.

In another preferred embodiment, the cortisone is continuously administered during a period of 24 hours. This can be done, for example, by means of typical infusion solutions.

In another preferred embodiment, the administration of the hydrocortisone is effected with the above-mentioned daily doses during a period of at least 2, preferably at least 5, particularly preferred at least 14 days, or until the sepsis is overcome.

It is finally preferred to supplement the above-illustrated combination therapy with selenium and corticoid by a supportive therapy with insulin which is in particular intended to adjust the blood sugar level such that 200 mg % are not exceeded.

The present examinations have shown a reduction of the mortality rate when patients were treated with the pharmaceutical preparation according to the invention. The reduction of the mortality rate of patients having a severe sepsis which were treated either with sodium selenite alone or with hydrocortisone alone or without any of these two active substances was examined, however, all of these groups received insulin as supportive therapy. The established data clearly show that the group who received selenium as well as hydrocortisone showed a clearly reduced mortality rate beyond the merely additive effect of the selenium and hydrocortisone effect. The data rather show a surprising synergistic effect of these two active substances with respect to the reduction of the mortality rate. The patients received either 1000 μg selenium per day or 200 mg hydrocortisone per day, or a combination thereof. The numbers in the columns show the number of patients per group.

The following examples will illustrate the invention.

Therapy approach according to the invention in case of severe sepsis:

The present invention is based on a prospective randomised study with patients having a severe sepsis and an Apache III score of more than 70 points. It was examined to what extent the mortality rate of these patients can be reduced with a combination therapy of sodium selenite and hydrocortisone, accompanied by a strict blood sugar adjustment by means of insulin. The patients were randomised and blinded, either with sodium selenite, in the form of one 1000 μg bolus per day, followed by further daily bolus injections with each 1000 μg sodium selenite for 14 days, or they were treated with a placebo. In addition, the patients received 35 μg sodium selenite per day as a basis. All patients additionally received hydrocortisone, 200 mg continuously over 24 hours, and this for the complete duration of the severe infection. The further medication including the administration of antibiotics corresponded to the usual practice. In addition, the blood sugar was adjusted with insulin such that it was below 200 mg %. The result was a reduction of the mortality rate by approx. 10-20% of the patients only treated with selenium, also by 10-20% of the patients only treated with hydrocortisone, however, with the combination of hydrocortisone/sodium selenite, the mortality rate of seriously ill septic patients could be reduced by 80% with the combination therapy (sodium selenite, hydrocortisone and blood sugar control). This result clearly shows the achievable synergistic effect with the combination therapy with sodium selenite and hydrocortisone with a simultaneous strict blood sugar adjustment with insulin.

1. A method of treating sepsis, systemic inflammatory response syndrome (SIRS), and/or septic shock in a patient, which method comprises administering to the patient (a) a pharmaceutical composition comprising a selenium-containing active substance, (b) a pharmaceutical composition comprising a corticoid-containing active substance, and (c) a pharmaceutical composition comprising insulin, whereby the patient is treated for sepsis, SIRS, and/or septic shock.

2. The method of claim 2, wherein at least 100 μg of selenium is administered per day.

3. The method of claim 3, wherein at least 1000 μg of selenium is administered per day.

4. The method of claim 3, wherein at least 3.34 mg sodium selenite×5 H2O is administered per day.

5. The method of claim 1, wherein the pharmaceutical composition comprising the selenium-containing active substance is administered by means of a bolus once a day.

6. The method of claim 1, wherein the pharmaceutical composition comprising the selenium-containing active substance is administered over a period of at least 7 days.

7. The method of claim 6, wherein the pharmaceutical composition comprising the selenium-containing active substance is administered over a period of at least 14 days.

8. The method of claim 1, wherein the pharmaceutical composition comprising the selenium-containing active substance is continuously administered over 24 hours.

9. The method of claim 5, wherein the pharmaceutical composition comprising the selenium-containing active substance is continuously administered over 24 hours.

10. The method of claim 1, wherein the corticoid-containing active substance is hydrocortisone.

11. The method of claim 10, wherein at least 50 mg hydrocortisone is administered per day.

12. The method of claim 11, wherein at least 200 mg hydrocortisone is administered per day.

13. The method of claim 10, wherein the pharmaceutical composition comprising the hydrocortisone is continuously administered over 24 hours.

14. The method of claim 10, wherein the pharmaceutical composition comprising the hydrocortisone is administered for at least 2 days.

15. The method of claim 14, wherein the pharmaceutical composition comprising the hydrocortisone is administered for at least 5 days.

16. The method of claim 1, wherein the blood sugar level of insulin in the patient does not exceed 200 mg %.
